(19) United States
(12) Patent Application Publication
(10) Pub. No.: US 2005/0186292 A1
(43) Pub. Date: Aug. 25, 2005

(54) Antimicrobial agent

(75) Inventors: Yoshihiro Fukuo, Tokyo (JP); Kazuya Ogasawara, Hayami-gun (JP); Katsuya Satoh, Hayami-gun (JP)

Correspondence Address: THE WEBB LAW FIRM, P.C., 700 KOPPERS BUILDING, 436 SEVENTH AVENUE, PITTSBURGH, PA, 15219, US

(73) Assignee: Japan Bio Science Laboratory Co., Ltd.

(21) Appl. No.: 11/061,190

(22) Filed: Feb. 18, 2005

(30) Foreign Application Priority Data
Feb. 20, 2004 (JP) 2004-44593

(51) Int. Cl. A61K 35/78

(52) U.S. Cl. 424/725; 424/779

(57) ABSTRACT

The present invention provides an antimicrobial agent against Helicobacter pylori, including a material derived from Angelica keiskei. It is preferable that the material derived from Angelica keiskei is a leaf, a stem or a root of Angelica keiskei or a processed product thereof, or sap of Angelica keiskei or a processed product thereof.

### BACKGROUND OF THE INVENTION

1. Field of the Invention

The present invention relates to an antimicrobial agent against Helicobacter pylori.

2. Description of the Related Art

Conventionally, medicines which protect gastric mucosa and/or suppress gastric acid secretion have been used to treat ulcers of digestive organs such as gastric ulcers and duodenal ulcers. For example, it is known that 2′-ethoxycarbonyl methoxy-4-n-hexyl-4′-(3-methyl-2-butenyloxy)chalcone, which is a chalcone derivative, protects gastric mucosa and/or suppresses gastric acid secretion (Japanese Laid-Open Patent Publication No. 3-163042). However, even if these pharmaceuticals which protect gastric mucosa and/or suppress gastric acid secretion are used, ulcers of digestive organs such as gastric ulcers and duodenal ulcers cannot be cured completely.

Regarding gastric ulcers and duodenal ulcers it is estimated that Helicobacter pylori (which used to be called Campylobacter pylori), which is a bacterium present in gastric mucosa, is one of the causes of the diseases (e.g., Journal of Clinical and Experimental Medicine (Igaku no Ayumi), vol. 159, pp. 795 (1991)). Furthermore, a correlation between Helicobacter pylori and reoccurrence of ulcers is suggested in some publications (e.g., The Lancet, vol. 336, pp. 755 (1990)). Therefore, antimicrobial agents against Helicobacter pylori are widely explored to treat and prevent ulcers of digestive organs such as gastric ulcers and duodenal ulcer.

As antimicrobial agents against Helicobacter pylori, various antibiotics such as penicillins, cephalosporin antibiotics, tetracycline antibiotics, and macrolide antibiotics have been examined. These antibiotics have high antimicrobial activities but have strong side effects such as hypersensitivity or diarrhea. Furthermore, long term use may cause serious side effects such as organopathy or hemopathy, and may cause resistant bacteria to appear. Benzimidazole compounds also have been examined as antimicrobial agents (Japanese Laid-Open Patent Publication No. 8-99808), but the antimicrobial activities of these compounds are weak.

For the purpose of preventing these problems due to long term use and reducing side effects, it has been attempted to use natural medications as antimicrobial agents. For example, it has been attempted to use phellodendron bark, magnolia bark, or cinnamon bark as an antimicrobial agent against Helicobacter pylori (Japanese Laid-Open Patent Publication No. 7-242560). However, the antimicrobial activities of these natural medications are not satisfactory.

Therefore, there is a demand for natural medications that have excellent antimicrobial activities against Helicobacter pylori.

### SUMMARY OF THE INVENTION

It is an object of the present invention to provide a substance having an excellent antimicrobial effect against Helicobacter pylori.

The present invention provides an antimicrobial agent against Helicobacter pylori, comprising a material derived from Angelica keiskei.

In a preferred embodiment, the material derived from Angelica keiskei is a leaf, a stem and a root of Angelica keiskei or a processed product thereof.

In another preferred embodiment, the material derived from Angelica keiskei is sap of Angelica keiskei or a processed product thereof.

Angelica keiskei is used widely as a health food and does not cause an adverse effect such as a side effect when it is ingested. Leaves, stems, roots or sap of Angelica keiskei, or their processed products and antimicrobial agents obtained from them have an excellent antimicrobial effect against Helicobacter pylori, even if the concentration is low. Therefore, the antimicrobial agent of the present invention can be utilized to treat and prevent ulcers of digestive organs such as gastric ulcers and duodenal ulcers as an antimicrobial agent that is highly safe and is excellent against Helicobacter pylori.

### DESCRIPTION OF THE PREFERRED EMBODIMENT

The antimicrobial agent against Helicobacter pylori of the present invention contains a material derived from Angelica keiskei. Angelica keiskei is a plant that belongs to the genus Angelica of the family Umbelliferae and is indigenous to Japan, and is generally called “ashitaba” in Japan. This plant is originally from Hachijo-shima, and grows in the Izu peninsula, Miura peninsula, Izu islands, Ohshima, etc., in addition to Hachijo-shima. This plant is commonly used as daily food of the habitants of Hachijo-shima, Izu islands, or the like. Angelica keiskei is a three-leaved plant, each leave being a large pinnate leaf, and has a large stem, and grows to a height of about 150 cm when it blooms with white flowers at the third year from the start of cultivation. Angelica keiskei is characterized by containing a large amount of yellow substances in its root, stem and leaves, and in that yellow juice exudes from its broken-out section. In this specification and the claims, “Angelica keiskei” includes plants belonging to the genus Angelica of the family Umbelliferae and having the above-described traits and characteristics.

Examples of materials derived from Angelica keiskei used in the present invention include roots, stems and leaves of Angelica keiskei, sap that has exuded from stems of Angelica keiskei (Angelica keiskei sap, Angelica keiskei yellow juice), and processed products thereof. These materials preferably can be obtained from Angelica keiskei before blooming after seeding and budding.

When the materials are leaves, stems, or roots of Angelica keiskei or processed products thereof, first, leaves, stems, or roots of Angelica keiskei are cut and dried to be turned into a dry powder. The leaves, stems, or roots of Angelica keiskei can be dried separately and after drying, they can be mixed. Alternatively, the leaves, stems, or roots of Angelica keiskei are mixed and then dried so as to obtain the dry powder. Alternatively, a ground product obtained by grinding the leaves, stems, or roots of Angelica keiskei separately or in combination, juice or juice residue of the ground products, and supernatant or residues obtained by centrifugation of the ground products can be used as a processed product. If necessary, a treatment such as concentration, drying, and shaping can be performed. The thus obtained materials derived from Angelica keiskei can be used as the antimicrobial agent without further treatment. In the present invention, the processed products include extracted products obtained by collecting a fraction having antimicrobial activities from the leaves, stems, roots or sap thereof, or dry powders thereof, using an appropriate solvent (e.g., water and organic solvents such as ethanol, acetone, and hexane).

Herein, there is no particular limitation regarding the drying method, and any methods such as forced-air drying, drying under reduced pressure and freeze drying can be used. Freeze drying is preferable in view of the stability of functional components in the antimicrobial agent and the solubility of the obtained dried product.

Shaping can be performed, if necessary. In general, an excipient can be added to the materials derived from Angelica keiskei so that a shaped product such as a powder, granules, or a tablet can be obtained. These shaped products can be prepared by a method commonly performed by those skilled in the art. As the excipient, for example, dextrin, lactose, crystalline cellulose, silicon dioxide, cyclic oligosaccharide or the like can be preferably used.

In general, the excipient can be mixed in 20 to 200 parts by weight with respect to 100 parts by weight (dry weight for liquid products) of the material derived from Angelica keiskei, although it depends on the material to which the excipient is to be added and the shaped product to be prepared.

The Angelica keiskei sap can be obtained by gathering leaves and stems of Angelica keiskei before blooming after seeding and budding and collecting yellow sap that is exuded from the stems. The Angelica keiskei sap contains chalcones which suppress gastric acid secretion. The amount of chalcones in the Angelica keiskei sap is far more than that contained in Angelica keiskei leaves. The Angelica keiskei sap can be used as the antimicrobial agent of the present invention without further treatments.

The Angelica keiskei sap can be optionally subjected to a treatment such as filtration or heating sterilization. As a heating sterilization treatment, any method commonly performed by those skilled in the art can be used, and for example, a vessel containing the sap can be immersed in hot water and left therein for several hours after the temperature of the sap in the vessel has reached the same temperature as the hot water or can be treated with an autoclave. The antimicrobial activity is not degraded by heating treatment so that heating sterilization treatment is useful for storage. The heating treatment may be performed to a mixture of the sap and an excipient as described below.

The Angelica keiskei sap contains a large amount of oil so that it is not turned into a powder simply by drying, and even with freeze drying, the Angelica keiskei sap aggregates and is difficult to shape into powder, granules, or tables. Therefore, an excipient is added to the Angelica keiskei sap, and the sap is dried by the drying methods as above (preferably, freeze drying) so that a processed product such as a powder can be obtained. Furthermore, an antimicrobial agent made of a processed product of the Angelica keiskei sap that is shaped into granules, tables or the like can be obtained.

There is no particular limitation regarding the mixture ratio of the Angelica keiskei sap and the excipient, and the mixture ratio can be determined in view of the antimicrobial activity. For example, they may be mixed at a weight ratio of 100:1 to 1:100. An appropriate solvent such as ethanol can be added for uniform mixture.

A pharmaceutical preparation containing the material derived from the Angelica keiskei obtained in the above-described manner can be used as an antimicrobial agent against Helicobacter pylori. The antimicrobial agent of the present invention can be utilized not only as a medicine, but also a health food product, a supplemental food product, or a functional food product. Furthermore, antimicrobial paper and antimicrobial sheets against Helicobacter pylori can be produced by applying or compounding it to a food wrapping paper, a wrapping sheet or the like.

Hereinafter, the present invention will be described by way of examples, but the present invention is not limited to these.

### EXAMPLES

### Example 1

The stems of Angelica keiskei originated from Hachijo-shima were cut at a suitable time after seeding and before blooming, and sap that has exuded from the stem was collected. The sap was filtrated and a vessel containing the filtrated liquid was immersed in boiled water (sterilization treatment) for 30 minutes so that the sterilized Angelica keiskei sap was obtained.

### Example 2

First, 70 g of the Angelica keiskei sap that had been subjected to a sterilization treatment in Example 1 and 30 g of a cyclodextrin powder were mixed uniformly, and the obtained mixture was subjected to freeze drying to obtain 45 g of a preparation containing the Angelica keiskei sap (hereinafter, referred to as “Angelica keiskei sap preparation”). This Angelica keiskei sap preparation contains components such as chalcones and coumarins.

### Example 3

The antimicrobial activity against Helicobacter pylori was examined using the obtained Angelica keiskei sap and the Angelica keiskei sap preparation. Angelica keiskei sap (a), (b) a five-fold dilution of Angelica keiskei sap (a) (dilution with sterilized water), (c) a suspension in which the Angelica keiskei sap preparation was suspended in a concentration of 200 mg/ml in a sterilized water, and (d) a five-fold dilution of the suspension (c) (the concentration of the Angelica keiskei sap preparation: 40 mg/ml) were prepared, and 50 μl of each of them was spotted in a sterilized disk having a diameter of 6.35 mm, and dried at 37° C. to obtain sample disks. Each sample disk was placed onto a Brain Heart Infusion blood agar medium containing 5% horse defibrinated blood to which Helicobacter pylori NCTC11637 was inoculated, and was cultured under microaerophilic conditions at 37° C. for 72 hours, and then the diameter of an inhibition circle appeared in the agar medium was measured. Two disks per sample were used. The results are shown in Table 1. As a positive control (e), a sterilized disk containing cephalothin (30 μg) was used, and as a negative control (f), those containing nalidixic acid (30 μg) was used (both manufactured by EIKEN CHEMICAL CO., LTD.).

TABLE 1

| | | | Diameter | |
|---|---|---|---|---|
| | | | of Inhibition | |
| | | | circle (mm) | |
| Sample | | Amount used | 1 | 2 |
| (a) | Angelica keiskei sap | Undiluted solution | 14 | 14 |
| (b) | Angelica keiskei sap | 5-fold dilution of | 11 | 12 |
| | | undiluted solution | | |
| (c) | Angelica keiskei sap preparation | 10 mg | 22 | 23 |
| (d) | Angelica keiskei sap preparation | 2 mg | 14 | 15 |
| (e) | Cephalothin | 30 μg | 44 | 46 |
| (f) | Nalidixic acid | 30 μg | 0 | 0 |

These results show that the antimicrobial activity against Helicobacter pylori was seen in both the Angelica keiskei sap and the Angelica keiskei sap preparation.

The composition of the Brain Heart Infusion blood agar medium (OXOID Ltd.) was obtained by adding horse defibrinated blood to the medium shown in Table 2 below so that the final concentration was 5 w/v %.

TABLE 2
Composition of medium

| | |
|---|---|
| Calf brain infusion | 12.5 g |
| Beef heart infusion | 5.0 g |
| Proteose pepton | 10.0 g |
| Sodium chloride | 5.0 g |
| Glucose | 2.0 g |
| Dibasic sodium phosphate | 2.5 g |
| Agar No. 1 | 10.0 g |
| Purified water | 1 L |
| (pH 7.4 ± 0.2) | |

The Brain Heart Infusion blood agar medium was prepared by first sterilizing the agar medium shown in Table 2, cooling to about 50° C., and then adding horse defibrinated blood so that the final concentration was 5 w/v %.

### Example 4

The Angelica keiskei sap (a) (undiluted solution) was diluted by a factor shown in Table 3 with the blood agar medium having the composition as above, so that the blood agar medium containing Angelica keiskei saps in various concentrations were prepared. Helicobacter pylori NCTC11637 was applied to the blood agar medium, and examined whether it grows or not. The results are shown in Table 3.

TABLE 3

| | | | | | | | | | |
|---|---|---|---|---|---|---|---|---|---|
| Dilution | 25 | 50 | 100 | 200 | 400 | 500 | 800 | 1600 | 3200 |
| factor of | | | | | | | | | |
| Angelica | | | | | | | | | |
| keiskei sap | | | | | | | | | |
| Growth of | − | − | − | − | − | + | + | + | + |
| Helicobacter | | | | | | | | | |
| pylori | | | | | | | | | |

+: growth,
−: no growth

As seen from Table 3, the antimicrobial activity against Helicobacter pylori was seen even in a 400-fold dilution of the Angelica keiskei sap.

### Example 5

Blood agar media containing the Angelica keiskei sap preparation in various concentrations shown in Table 4 were prepared using a suspension containing the Angelica keiskei sap preparation (c) in a concentration of 200 mg/ml. Helicobacter pylori NCTC11637 was inoculated on the blood agar medium, and the minimum growth inhibition concentration (MIC) was measured. The results are shown in Table 4.

TABLE 4

| | | | | | | | | | |
|---|---|---|---|---|---|---|---|---|---|
| Concentration | 40 | 20 | 4 | 2 | 0.5 | 0.4 | 0.25 | 0.125 | 0.0625 |
| of Angelica | | | | | | | | | |
| keiskei sap | | | | | | | | | |
| preparation | | | | | | | | | |
| (mg/ml) | | | | | | | | | |
| Growth of | − | − | − | − | − | + | + | + | + |
| Helicobacter | | | | | | | | | |
| pylori | | | | | | | | | |

+: growth,
−: no growth

As shown in Table 4, the antimicrobial activity against Helicobacter pylori was seen even in the Angelica keiskei sap preparation in a concentration of 0.5 mg/ml (MIC: 0.5 mg/ml). Thus, it has been shown that the preparation containing the material derived from Angelica keiskei has antimicrobial activity against Helicobacter pylori.

The invention may be embodied in other forms without departing from the spirit or essential characteristics thereof The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

1. An antimicrobial agent against Helicobacter pylori, comprising a material derived from Angelica keiskei.

2. The antimicrobial agent according to claim 1, wherein the material derived from Angelica keiskei is a leaf, a stem or a root of Angelica keiskei or a processed product thereof.

3. The antimicrobial agent according to claim 1, wherein the material derived from Angelica keiskei is sap of Angelica keiskei or a processed product thereof.
